(19) United States
(12) Patent Application Publication
(10) Pub. No.: US 2026/0130593 A1
(43) Pub. Date: May 14, 2026

(54) HYBRID CONNECTOR FOR INTRAVASCULAR IMAGING DEVICES

(71) Applicant: Boston Scientific Scimed, Inc., Maple Grove, MN (US)

(72) Inventor: Andrew Brian Graveley, Shoreview, MN (US)

(73) Assignee: Boston Scientific Scimed, Inc., Maple Grove, MN (US)

(21) Appl. No.: 19/386,762

(22) Filed: Nov. 12, 2025

Related U.S. Application Data
(60) Provisional application No. 63/719,821, filed on Nov. 13, 2024.

(51) Int. Cl. A61B 5/00

(52) U.S. Cl. CPC A61B 5/0084; A61B 5/0062; A61B 5/6852; A61B 2560/0462; A61B 2562/227; A61B 2562/228

(57) ABSTRACT

An intravascular imaging system includes an imaging catheter, a first imaging device that is translatable and rotatable within the imaging catheter and a second imaging device that is translatable and rotatable within the imaging catheter. A connector assembly is disposed adjacent to a proximal end region of the imaging catheter and is configured to connect the imaging catheter to a control unit. The connector assembly includes a first pair of electrical connectors for connecting the first imaging device to the control unit and a second pair of electrical connectors for connecting the second imaging device to the control unit. The imaging catheter may further include an optical imaging device and an optical fiber cable that extends between the optical imaging device and the connector assembly.

### CROSS-REFERENCE TO RELATED APPLICATIONS

This application claims the benefit of priority under 35 U.S.C. §119 of U.S. Provisional Application No. 63/719,821, filed November 13, 2024, the entire disclosure of which is hereby incorporated by reference.

### TECHNICAL FIELD

The present disclosure pertains to medical devices, and methods for manufacturing medical devices. More particularly, the present disclosure pertains to intravascular imaging devices.

### BACKGROUND

A wide variety of medical devices have been developed for medical use, for example, intravascular use. Some of these devices include guidewires, catheters, and the like. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### SUMMARY

This disclosure provides design, material, manufacturing method, and use alternatives for medical devices. An example may be found in an intravascular imaging system. The intravascular imaging system includes an imaging catheter, a first imaging device translatable and rotatable within the imaging catheter and a second imaging device translatable and rotatable within the imaging catheter. A first connector assembly is disposed adjacent to a proximal end region of the imaging catheter and is configured to mechanically connect the imaging catheter to a control unit. The first connector assembly includes a first pair of electrical connectors for electrically connecting the first imaging device to the control unit and a second pair of electrical connectors for electrically connecting the second imaging device to the control unit.

Alternatively or additionally, the first imaging device may include an ultrasound transducer.

Alternatively or additionally, the intravascular imaging system may further include an optical imaging device translatable and rotatable within the imaging catheter.

Alternatively or additionally, the intravascular imaging system may further include an optical fiber that extends between the optical imaging device and the connector assembly.

Alternatively or additionally, the first connector assembly may further include a ferrule and a connecting sleeve disposed about the ferrule.

Alternatively or additionally, the intravascular imaging system may further include a control unit.

Alternatively or additionally, the control unit may include a second connector assembly that is adapted to mechanically and electrically engage the first connector assembly.

Alternatively or additionally, the first connector assembly may include a plurality of connector pins and the second connector assembly may include a plurality of pin receptacles that are each adapted to receive a corresponding one of the plurality of connector pins.

Alternatively or additionally, the intravascular imaging system may further include a second fiber optic cable extending through the control unit.

Alternatively or additionally, the control unit may include a control unit housing.

Alternatively or additionally, the control unit housing may include a base section adapted to secure the control unit to a sled.

Alternatively or additionally, the control unit may further include a rotary motor that is disposed within the control unit housing such that the rotary motor is outside of the base section of the control unit housing.

Alternatively or additionally, the control unit may include a first common mode choke that is associated with the first pair of electrical connectors and a second common mode choke that is associated with the second pair of electrical connectors.

Another example may be found in an intravascular imaging system. The intravascular imaging system includes an imaging catheter and an imaging core that is translatable and rotatable within the imaging catheter. A connector assembly is disposed adjacent to a proximal end region of the imaging catheter and is configured to connect the imaging catheter to a control unit. The connector assembly includes a first pair of electrical connectors for mechanically coupling a first imaging device to the control unit, a second pair of electrical connectors for electrically coupling a second imaging device to the control unit, and an optical connector for optically coupling an optical imaging device to the control unit.

Alternatively or additionally, the imaging core may include the first imaging device, the second imaging device, and the optical imaging device.

Alternatively or additionally, the control unit may include a first common mode choke that is associated with the first pair of electrical connectors and a second common mode choke that is associated with the second pair of electrical connectors.

Alternatively or additionally, the control unit may further include a control unit housing having a base section and a rotary motor that is disposed outside of the base section of the control unit housing.

Another example may be found in an intravascular imaging system. The intravascular imaging system includes an imaging catheter, a control unit and a connector assembly that is disposed adjacent to a proximal end region of the imaging catheter and that is configured to mechanically connect the imaging catheter to the control unit. The control unit includes a control unit housing, a control unit housing base section that is adapted to be releasably secured to a sled, and a rotary motor that is disposed within the control unit housing outside of the control unit housing base section. The connector assembly includes a first pair of electrical connectors for electrically connecting a first imaging device to the control unit and a second pair of electrical connectors for electrically connecting a second imaging device to the control unit.

Alternatively or additionally, the rotary motor may have a length that is greater than a diameter of the control unit housing base section.

Alternatively or additionally, the control unit may include a first common mode choke that is associated with the first pair of electrical connectors and a second common mode choke that is associated with the second pair of electrical connectors.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 is a partially cutaway side view of an illustrative medical device;

FIG. 2 is a side view of an illustrative medical device;

FIG. 3 is a perspective view of a portion of an illustrative imaging core;

FIG. 4 is a perspective view of a portion of an illustrative medical device system;

FIG. 5 is a perspective view of a portion of an illustrative medical device system;

FIG. 6 is a perspective view of a portion of an illustrative medical device system;

FIG. 7 is a perspective view of a portion of an illustrative medical device system;

FIG. 8 is a perspective view of a portion of an illustrative medical device system; and

FIG. 9 is a perspective view of a portion of an illustrative medical device system.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (e.g., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used connection with other embodiments whether or not explicitly described unless clearly stated to the contrary.

In some instances, an intravascular imaging system may include an imaging catheter, a first imaging device that is translatable and rotatable within the imaging catheter and a second imaging device that is translatable and rotatable within the imaging catheter. A first connector assembly is disposed adjacent to a proximal end region of the imaging catheter and is configured to electrically connect the imaging catheter to a control unit. The first connector assembly includes a first pair of electrical connectors for connecting the first imaging device to the control unit and a second pair of electrical connectors for connecting the second imaging device to the control unit.

In some cases, the first imaging device may include an ultrasound transducer. The intravascular imaging system may further include an optical imaging device, for example, that is translatable and rotatable within the imaging catheter. In some cases, an optical fiber cable may extend between the optical imaging device and the first connector assembly. The first connector assembly may further include a ferrule and a connecting sleeve disposed about the ferrule.

In some cases, the intravascular imaging system may include a control unit. The control unit may include a second connector assembly that is adapted to mechanically and electrically engage the first connector assembly. In some cases, the first connector assembly may include a plurality of connector pins and the second connector assembly may include a plurality of pin receptacles, where each of the plurality of pin receptacles are adapted to receive a corresponding one of the plurality of connector pins. A second fiber optic cable may extend through the control unit. In some cases, the control unit may include a control unit housing including a base section that is adapted to secure the control unit to a sled. In some cases, the control unit may further include a rotary motor that is disposed within the control unit housing such that the rotary motor is outside of the base section of the control unit housing. In some cases, the control unit may include a first common mode choke that is associated with the first pair of electrical connectors and a second common mode choke that is associated with the second pair of electrical connectors.

In some instances, an intravascular imaging system includes an imaging catheter and an imaging core that is translatable and rotatable within the imaging catheter. A connector assembly is disposed adjacent to a proximal end region of the imaging catheter and is configured to connect the imaging catheter to a control unit. The connector assembly includes a first pair of electrical connectors for electrically coupling a first imaging device to the control unit, a second pair of electrical connectors for electrically coupling a second imaging device to the control unit, and an optical connector for optically coupling an optical imaging device to the control unit.

In some cases, the imaging core may include the first imaging device, the second imaging device, and the optical imaging device. In some cases, the control unit may include a first common mode choke that is associated with the first pair of electrical connectors and a second common mode choke that is associated with the second pair of electrical connectors. In some cases, the control unit may further include a control unit housing having a base section and a rotary motor that is disposed outside of the base section of the control unit housing.

In some instances, an intravascular imaging system includes an imaging catheter and a control unit. The control unit includes a control unit housing, a control unit housing base section adapted to be releasably secured to a sled, and a rotary motor that is disposed within the control unit housing outside of the control unit housing base section. A connector assembly is disposed adjacent to a proximal end region of the imaging catheter and is configured to connect the imaging catheter to the control unit. The connector assembly includes a first pair of electrical connectors for connecting a first imaging device to the control unit and a second pair of electrical connectors for connecting a second imaging device to the control unit.

In some cases, the rotary motor has a length that is greater than a diameter of the control unit housing base section. In some cases, the control unit may include a first common mode choke that is associated with the first pair of electrical connectors and a second common mode choke that is associated with the second pair of electrical connectors.

The following detailed description should be read with reference to the drawings in which similar elements in different drawings are numbered the same. The drawings, which are not necessarily to scale, depict illustrative embodiments and are not intended to limit the scope of the invention.

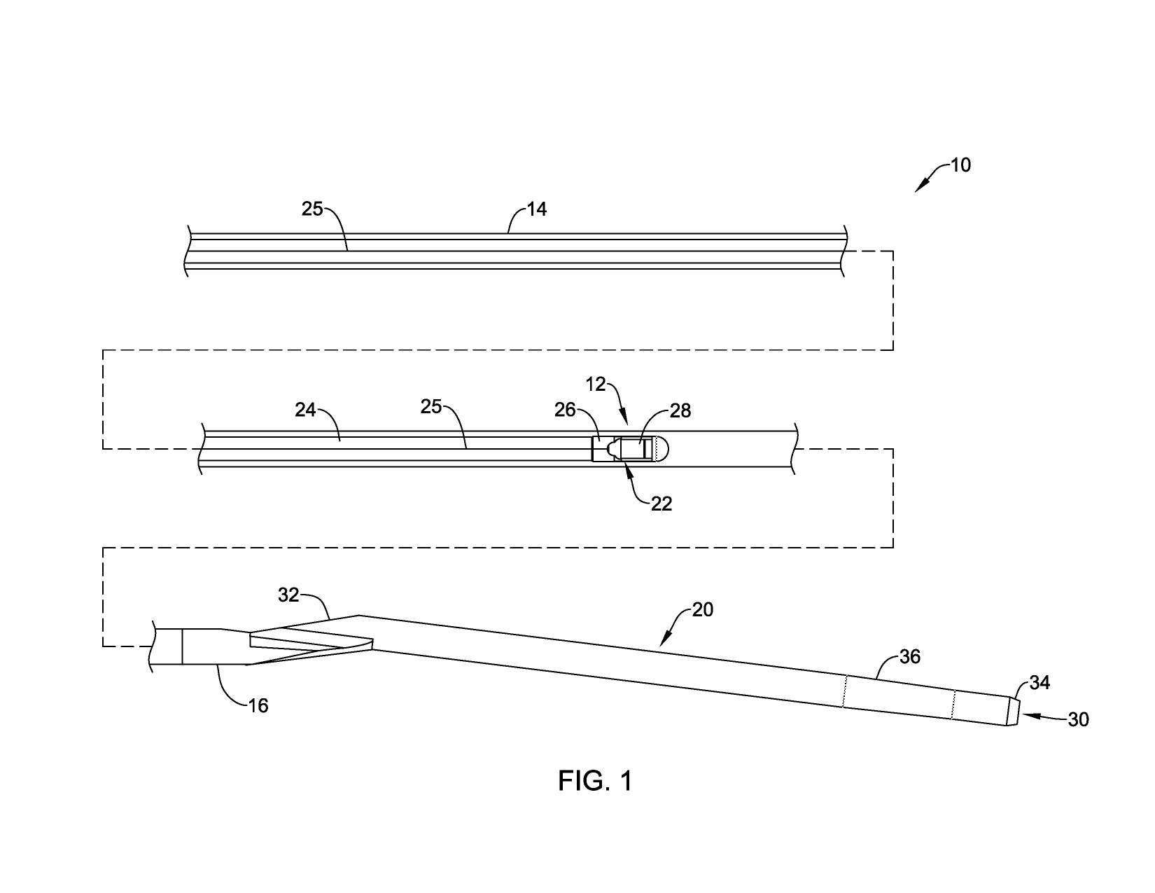

FIG. 1 is a side view of a portion of an illustrative medical device 10. In at least some instances, the medical device 10 takes the form of an imaging medical device. For example, the medical device 10 may be an intravascular ultrasound (IVUS) device that may be used to image a blood vessel. In some of these and in other instances the medical device 10 may be an optical coherence tomography (OCT) imaging device, a near-infrared spectroscopy (NIRS) imaging device, near-infrared fluorescence (NIRF) imaging device, a photoacoustic imaging device, a fluorescence-lifetime imaging device, combinations thereof (including combinations that include IVUS), and/or the like. In addition to be used for intravascular imaging, the medical device 10 may also be used for pulmonary procedures/imaging. The structure/form of the medical device 10 can vary. In some instances, the medical device 10 may include an elongate shaft 12 having a proximal end region 14 and a distal end region 16. A tip member 20 may be coupled to or otherwise disposed adjacent to the distal end region 16. The tip member 20 may include a guidewire lumen 30 having a guidewire exit port 32, an atraumatic distal end 34, one or more radiopaque markers 36, and/or other features. In some embodiments, the tip member 20 may extend at a non-parallel angle to the proximal end region 14 of the elongate shaft 12.

An imaging assembly 22 (e.g., which may sometime be referred to as an imaging core) may be disposed within a lumen of the elongate shaft 12. In general, the imaging core 22 may be used to capture/generate images of a blood vessel. In some instances, the medical device may include devices and/or features similar to those disclosed in U.S. Patent Application Pub. No. US 2012/0059241 and U.S. Patent Application Pub. No. US 2017/0164925, the entire disclosures of which are herein incorporated by reference. In at least some instances, the medical device 10 may resemble and/or include features that resemble the OPTICROSS™ Imaging Catheter, commercially available from BOSTON SCIENTIFIC, Marlborough, MA.

The imaging core 22 may include a drive shaft or cable 24, a housing 26, and an imaging member or transducer 28 coupled to the drive shaft 24 and/or housing 26. In at least some instances, the transducer 28 includes an ultrasound transducer. Other transducers are also contemplated. The transducer 28 may be rotatable and/or axially translatable relative to the elongate shaft 12. For example, the drive shaft 24 may be rotated and/or translated in order to rotate and/or translate the transducer 28 (and the housing 26). A conductor 25 may be coupled to the transducer 28 and extend proximally therefrom. In some instances, the conductor 25 may take the form of a wire or cable (e.g., a coaxial cable) with suitable electrical conduction properties that allow the conductor 25 to energize the transducer 28. In some of these and in other instances, the conductor 25 may include a chip that is configured to amplify, multiplex or beamform signals delivered to and received by the imaging core 22.

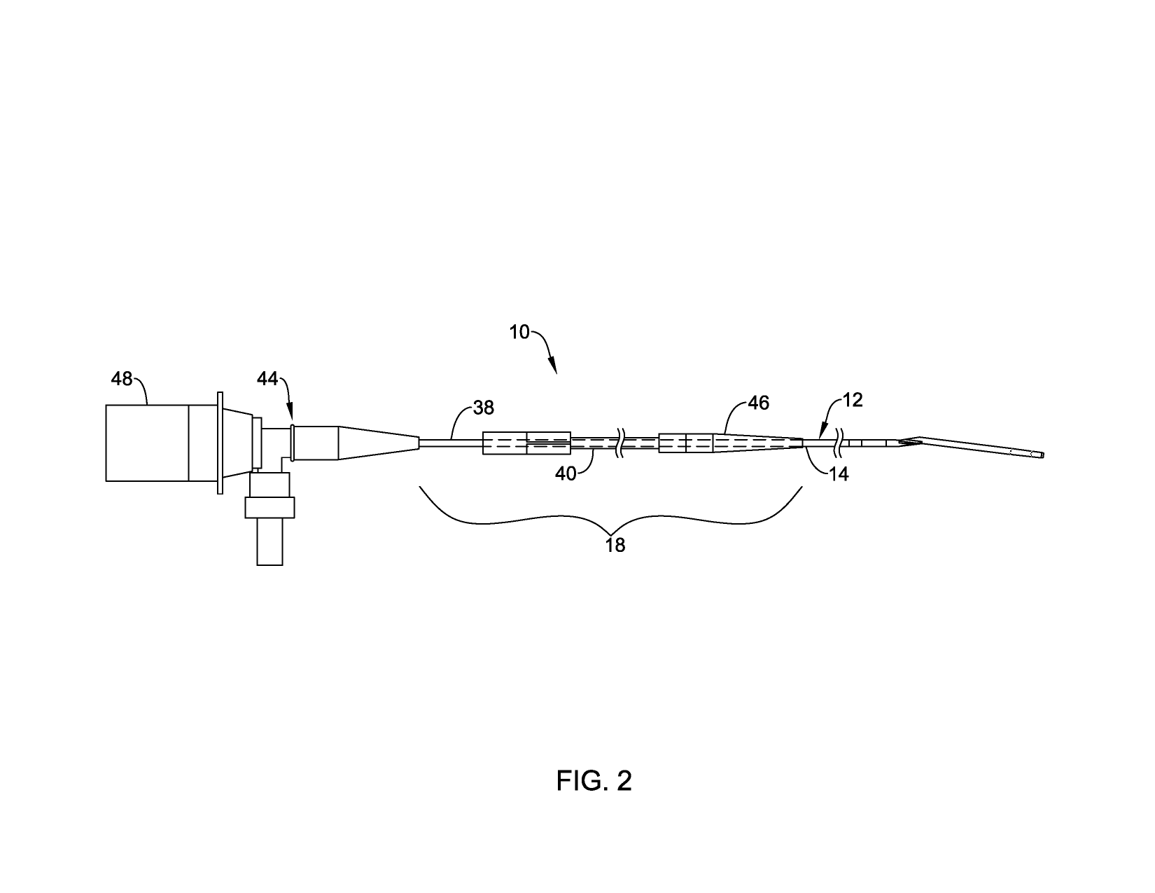

The proximal end region 14 of the elongate shaft 12 may be coupled to a telescoping assembly 18 as shown in FIG. 2, which is a side view of an illustrative medical device 10. In general, the telescoping assembly 18 may be configured to allow the medical device operator to move the drive shaft 24 including the imaging core 22 proximally and distally within the catheter (e.g., relative to the elongate shaft 12), without having to move the entire catheter within the patient. This allows the catheter operator to easily change the location of the imaging core 22 within the patient. For example, the telescoping assembly 18 may be actuated to change the location of the imaging core 22 within the elongate shaft 12.

The proximal end region 14 of the elongate shaft 12 may be coupled to the telescoping assembly 18. For example, the proximal end region 14 of the elongate shaft 12 may be coupled to a distal hub 46 of the telescoping assembly 18. A proximal hub 44 may be coupled to the telescoping assembly 18 (e.g., at the proximal end of the telescoping assembly 18). The drive shaft 24 (see FIG. 1) may extend through the telescoping assembly 18 and be coupled to and/or otherwise secured to the proximal hub 44. The proximal hub 44 may include a connector assembly 48. In general, the connector assembly 48 may allow the medical device 10 (e.g., the elongate shaft 12) to connect to a control unit (e.g., a motor drive unit and/or the like) as described in more detail herein.

The telescoping assembly 18 may include a first sheath 38 and a second sheath 40. In some instances, the first sheath 38 may be understood to be an inner telescoping tube 38 and the second sheath 40 may be understood to be an outer telescoping tube 40. Generally, the outer telescoping tube 40 may be disposed over the inner telescoping tube 38. The inner telescoping tube 38 may be coupled to or otherwise secured to the proximal hub 44. The outer telescoping tube 40 may be coupled or otherwise secured to the distal hub 46. The inner telescoping tube 38 may be axially and/or rotatably moveable relative to the outer telescoping tube 40. Because the drive shaft 24 may be secured to the proximal hub 44 and/or the inner telescoping tube 38 and because the elongate shaft 12 may be secured to the distal hub 46, movement of the proximal hub 44 relative to the distal hub 46 results in movement of the inner telescoping tube 38 and the drive shaft 24 relative to the distal hub 46 and/or the elongate shaft 12.

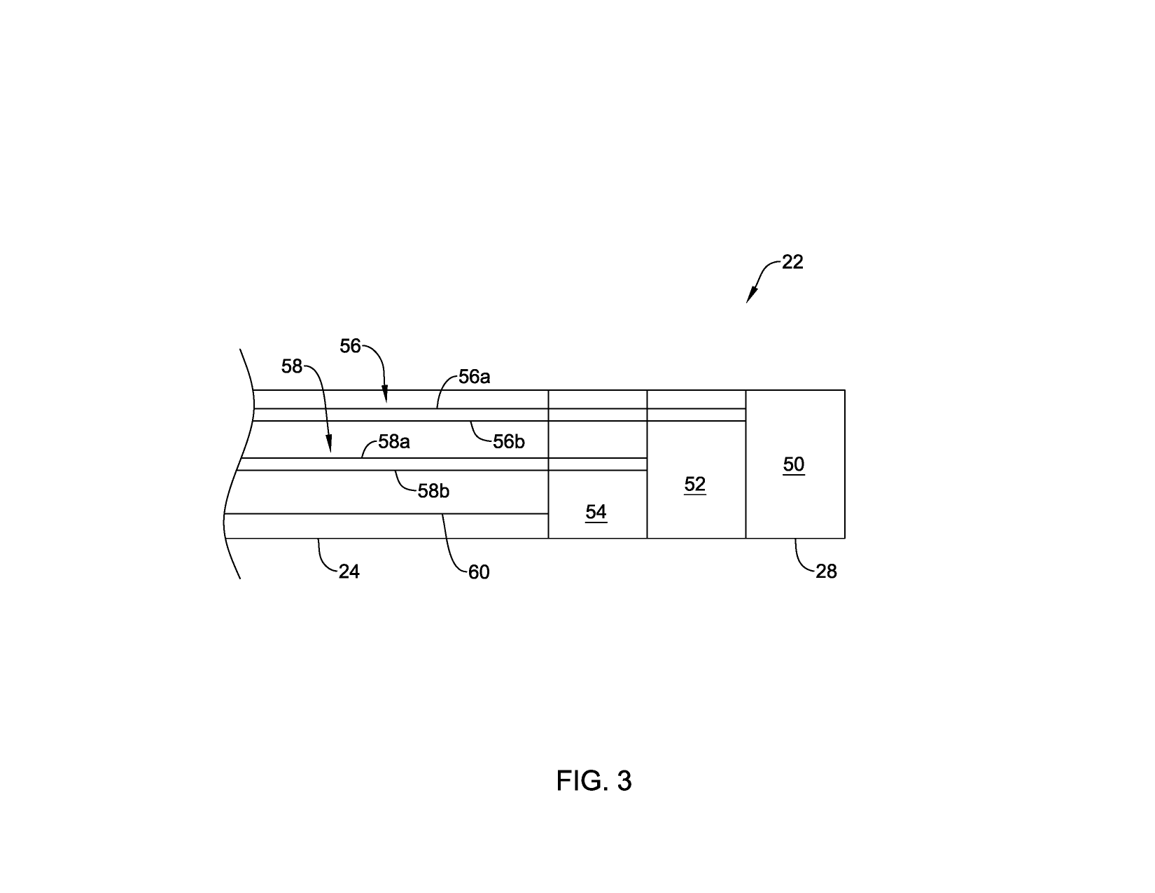

For some medical interventions, it may be desirable to have more than one type of imaging modality. For example, the imaging core 22 may include two or more distinct devices. FIG. 3 is a schematic view of a portion of the imaging core 22 that includes three distinct devices. As shown for example in FIG. 3, the imaging core 22 may include a first imaging device 50, a second imaging device 52 and a third imaging device 54. As an example, the first imaging device 50 may be an ultrasound device and thus may include the ultrasound transducer 28. The second imaging device 50 may be any of a variety of different imaging devices such as one or more of an optical coherence tomography (OCT) imaging device or a NIRF imaging device that can signify fluorescence intensity as a function of angular position. In some cases, such information may be considered to be one-dimensional (e.g., the depth of the fluorescent signal within the tissue is unknown). Thus, the second imaging device 50 may provide additional fluorescent information that helps to enhance the two-dimensional IVUS image and to improve interpretation of the two-dimensional IVUS image, rather than providing a separate two-dimensional image. Other imaging devices are contemplated including those disclosed herein. In some cases, another imaging modality may show features that are more difficult to see in the IVUS image. In some cases, the third imaging device 54 may be an optical imaging device.

The first imaging device 50 may be adapted to be electrically operated and/or to provide an electrical signal. In some cases, the first imaging device 50 may be operably coupled with a first pair 56 of electrical connectors, individually labeled as 56a and 56b, that are individual wires. In some cases, one of the wires is intended to conduct signal and another of the wires are intended to act as ground. The first pair 56 of electrical connectors electrically couple the first imaging device 50 with a control unit. The first pair 56 of electrical connectors pass through or around the second imaging device 52 and the third imaging device 54, and as such are not electrically coupled to either of the second imaging device 52 or the third imaging device 54. The second imaging device 52 may be adapted to be electrically operated and/or to provide an electrical signal. In some cases, the second imaging device 52 may be operably coupled with a second pair 58 of electrical connectors, individually labeled as 58a and 58b, that are individual wires. In some cases, one of the wires is intended to conduct a signal while the other acts as ground. The second pair 58 of electrical connectors electrically couple the second imaging device 52 with a control unit. The second pair 58 of electrical connectors pass through or around the third imaging device 54, and as such are not electrically coupled to the third imaging device 54. As noted, in some cases, the third imaging device 54 may be an optical imaging device, and as such may be operably coupled with a fiber optic cable 60.

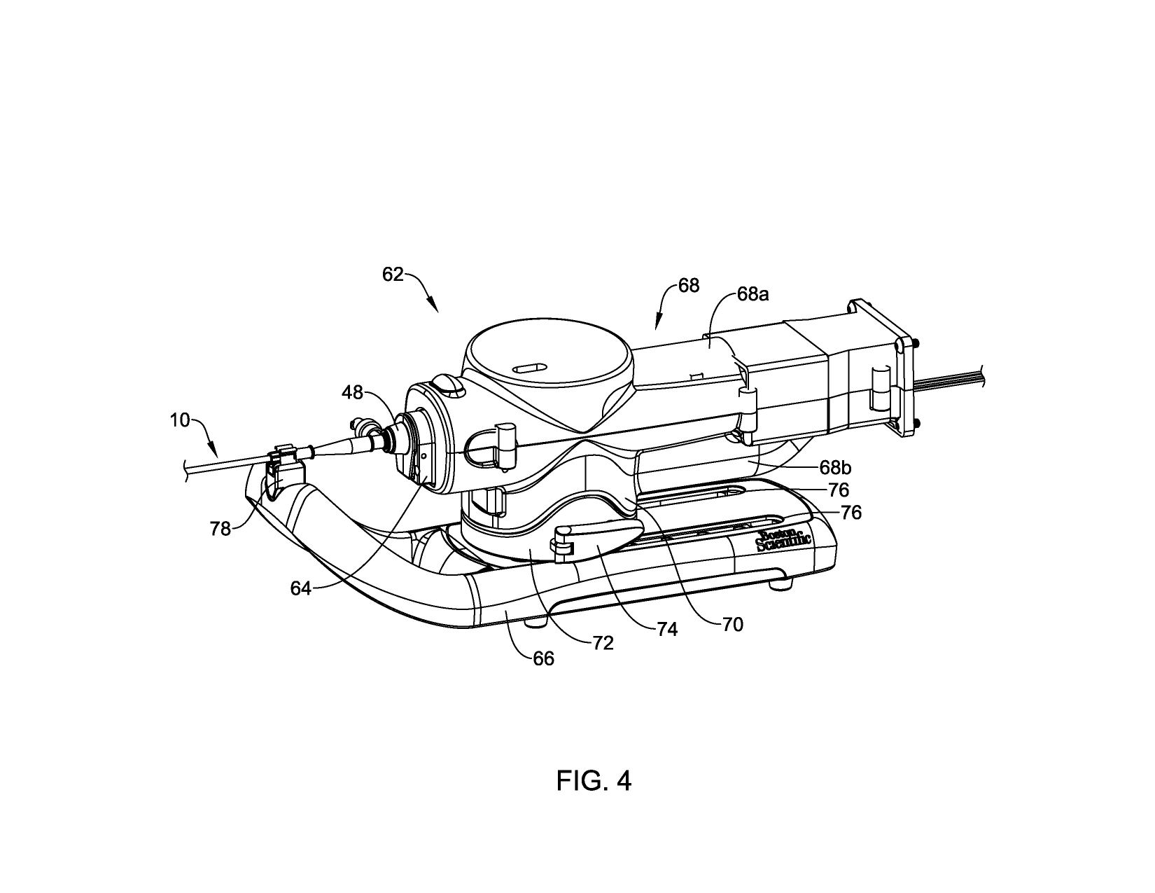

It will be appreciated that in devices such as the medical device 10, the imaging core 22 may be rotated and translated relative to the shaft 12. In order to do so, the shaft 12 may be connected to a control unit such as a motor drive unit (e.g., a motor drive unit 62 as shown in FIG. 4. For example, FIG. 4 is a perspective view showing the medical device 10 coupled to a motor drive unit 62. In some cases, the connector assembly 48 may be coupled to a connector receptacle 64 on the motor drive unit 62. When doing so, the connector assembly 48 and the connector receptacle 64 may be coupled to the motor drive unit 62 in a manner that permits the rotation and translation of the imaging core 22 as well as the ability to power and energize each of the first imaging device 50, the second imaging device 52 and the third imaging device 54. At least some details regarding the connections between the connector assembly 48 and the connector receptacle 64 that allow for rotation and translation, as well as power and energy transmission are disclosed herein.

In some cases, as shown, the motor drive unit 62 may be slidingly mounted on a sled 66. The motor drive unit 62 may be considered as including a housing 68 that may include an upper housing portion 68a and a lower housing portion 68b. In some cases, the housing 68 includes a base section 70 that may be adapted to be releasably secured to a sled cup 72 that may be molded as part of the sled 66, for example. In some cases, the base section 70 may be releasably secured to the sled cup 72 via a sled cup latch 74. The sled cup 72 may be adapted to slide along the sled 66, guided by slots 76. In some cases, the medical device 10 may be adapted to a site within the vasculature, for example, that is at a distal edge or just past a distal edge of a desired location for visualization. The medical device 10 may be pulled proximally in a pullback procedure to view the desired location as the medical device 10 (and hence the imaging core 22) is pulled proximally past where the desired location is. To accomplish the pullback, the medical device 10 may be moved manually. In some cases, the motor drive unit 62 (with the medical device 10 attached thereto) may be mechanically pulled back relative to the sled 66 via an external unit (not shown). The sled 66 may include a catheter cradle region 78 that may be configured to help support the medical device 10 and/or help to keep the elongate shaft 12 stationary during a pullback procedure.

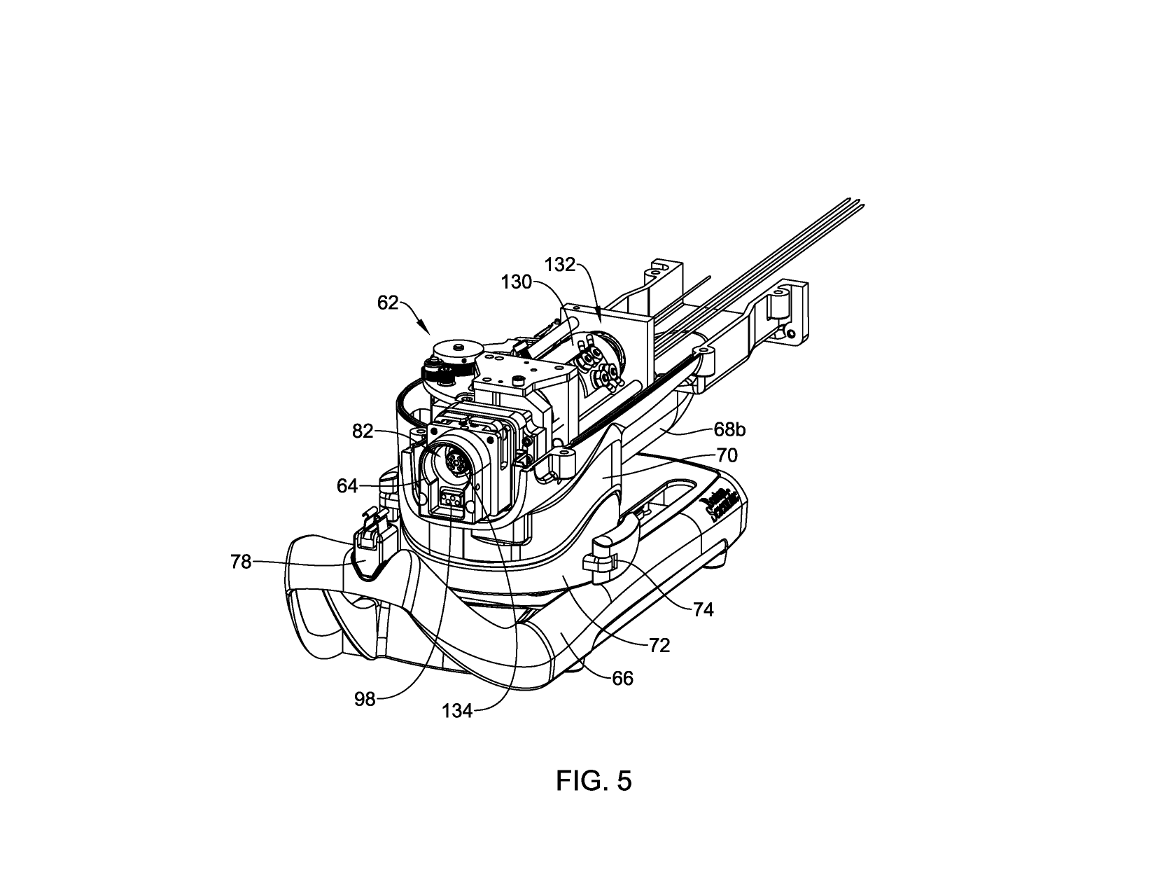

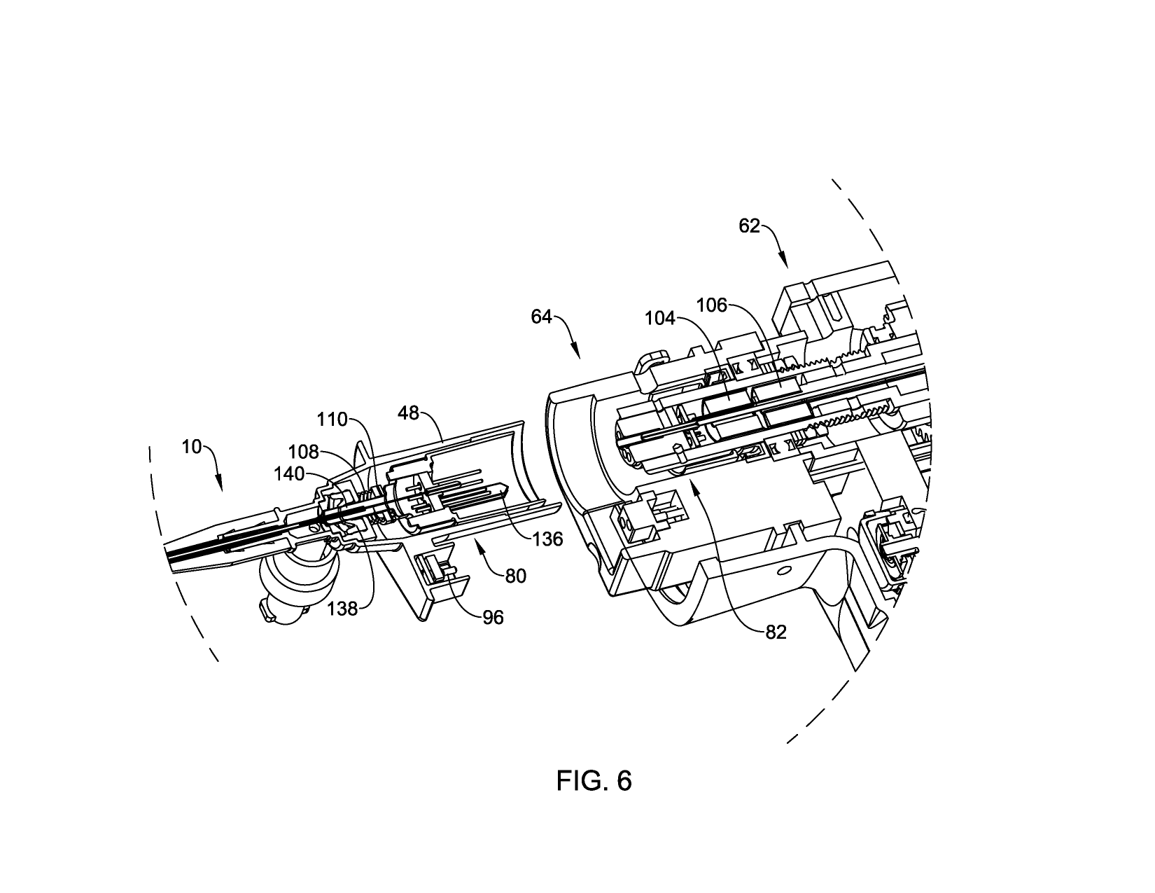

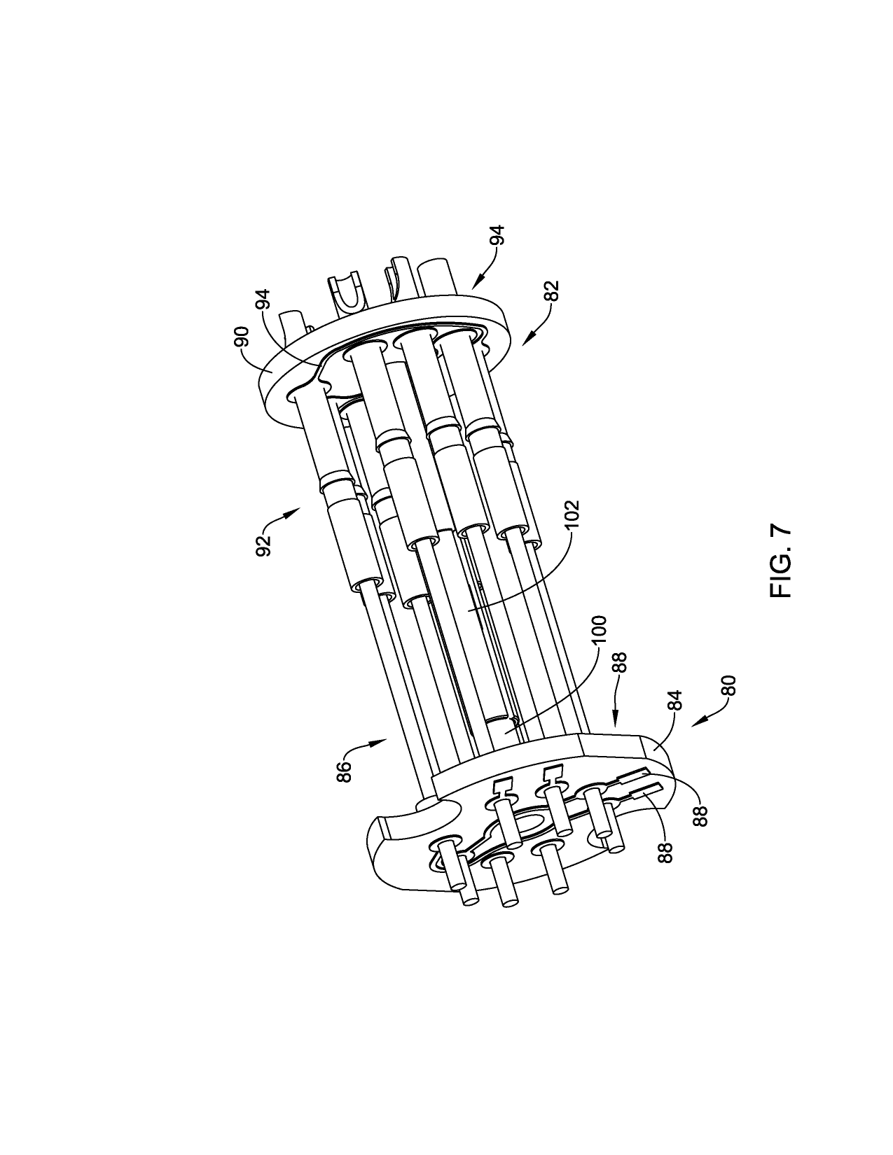

FIG. 5 is a partial cross-sectional view of the motor drive unit 62 with the medical device 10 disconnected therefrom while FIGS. 6, 8 and 9 are partial cross-sectional views of the motor drive unit 62 coupled with the medical device 10. The connector receptacle 64 is generally configured to receive the connector assembly 48 of the medical device 10. As best seen in FIG. 6, the medical device 10 includes a first connector assembly 80 and the motor drive unit 62 includes a second connector assembly 82 that is adapted to mechanically and electrically couple to the first connector assembly 80 in order to couple the medical device 10 to the motor drive unit 62. The first connector assembly 80 and the second connector assembly 82 are mechanically releasable. Because FIG. 6 is a cross-sectional view, only part of the first connector assembly 80 and the second connector assembly 82 are visible. FIG. 7 is a perspective view showing the first connector assembly 80 engaged with the second connector assembly 82.

As seen in FIG. 7, the first connector assembly 80 includes a printed circuit board (PCB) 84. A total of eight electrical pins 86 extend proximally from the PCB 84 and are electrically coupled to the PCB 84. In some cases, the PCB 84 may be coupled to the drive shaft 24. The first connector assembly 80 may be considered as being part of the medical device 10. In some cases, the first connector assembly 80 may be considered as providing eight distinct electrical connections. In some cases, the PCB 84 may include traces 88 that electrically couple each of the electrical pins 86 together. Two traces 88 are visible on one side of the PCB 84, and there may be two additional traces 88 on an opposing side of the PCB 84. As a result, the eight electrical pins 86 may be considered as representing a total of four electrical connectors, or two pairs of electrical connectors corresponding to the first pair 56 of electrical connectors and the second pair 58 of electrical connectors referenced for example in FIG. 3.

The second connector assembly 82 includes a PCB 90. A total of eight pin receptacles 92 extend distally from the PCB 90 and are electrically coupled to the PCB 90. Each of the pin receptacles 92 are adapted to receive a corresponding one of the electrical pins 86. The second connector assembly 82 may be considered as being part of the motor drive unit 62. In some cases, the second connector assembly 82 may be considered as providing eight distinct electrical connections. In some cases, the PCB 90 may include traces 94 that electrically couple each of the pin receptacles 92 together. This means that the first connector assembly 80 may be coupled with the second connector assembly 82 in either of two relative rotational positions that are 180 degrees apart. Even if the connector assembly 48 may only be connected to the motor drive unit 62 in a single orientation, particularly since the connector assembly 48 also includes a secondary electrical coupler 96 (FIG. 6) that is adapted to electrically engage a corresponding secondary electrical coupler 98 (FIG. 5) included with the motor drive unit 62, it is important to note that the first connector assembly 80 is rotatable within the connector assembly 48 and the second connector assembly 82 is rotatable within the motor drive unit 62. Thus, allowing for two equivalent coupling positions 180 degrees apart may make it easier for the first connector assembly 80 to engage the second connector assembly 82.

In some cases, as shown in FIG. 5, the motor drive unit 62 may include a cam surface 134 that can facilitate properly aligning the first connector assembly 80 with the second connector assembly 82. In some cases, the first connector assembly 80 may include a member 136 (shown in FIG. 6) that has a tapered end adapted to engage the cam surface 134. As the first connector assembly 80 is urged towards the second connector assembly 82, the tapered end of the member 136 engages the cam surface 134 and automatically moves the first connector assembly 80 into one of two orientations 180 degrees apart that allow the first connector assembly 80 to easily engage the second connector assembly 82. As seen in FIG. 6, the medical device 10 includes a fluid seal 138 that provides a fluid seal against a long cylindrical section 140 to prevent fluid leakage. The cylindrical section 140 allows for axial movement of the first connector assembly 80 while still preventing leaks.

While a total of eight electrical pins 86 and a corresponding total of eight pin receptacles 92 are shown, it will be appreciated that in some cases, there may be more or less than eight electrical pins 86 and pin receptacles 92. For example, if there is a desire to only electrically couple the motor drive unit 62 to a single imaging device (such as the first imaging device 50 or the second imaging device 52 as shown in FIG. 3, there may be only four electrical pins 86 and pin receptacles 92. If there is a desire to electrically couple the motor drive unit 62 to three distinct imaging devices also needing an electrical connection, the first connector assembly 80 may include twelve electrical pins 86 and the second connector assembly 82 may include twelve pin receptacles 92. The relative number of electrical pins 86 and pin receptacles 92 may also vary, depending on whether any of the electrical pins 86 and pin receptacles 92 are electrically shorted together to provide for two or more equivalent coupling positions.

In some cases, the relative spacing between each of the electrical pins 86 is selected to make the characteristic impedance of the electrical path through the electrical pins 86 to more closely match an impedance of the rest of the system. In some cases, the impedance is about 50 Ohms. In some cases, both the spacing between conductor pairs and the relative permittivity of the dielectric material (including the PCB 84) between the conductor pairs should be considered in order to get a better match of the characteristic impedance. Characteristic impedance is relevant because changes in this property along the length of the transmission path may generate reflections that can cause the signal to weaken and lose integrity.

The first connector assembly 80 includes a ferrule 100 (FIG. 7) and a connecting sleeve 102 that is disposed about the ferrule 100. The ferrule 100 provides a connection with the optical fiber cable 60 (FIG. 3). With particular reference to FIG. 6, the motor drive unit 62 may include a first common mode choke 104 and a second common mode choke 106. Each of the first common mode choke 104 and the second common mode choke 106 may be a ferrite choke, for example. Each of the first common mode choke 104 and the second common mode choke 106 may be located elsewhere within the housing 68. In some cases, the first common mode choke 104 may be associated with the first pair 56 of electrical connectors (FIG. 3) and the second common mode choke 106 may be associated with the second pair 58 of electrical connectors. In some cases, the first common mode choke 104 may instead be associated with the second pair 58 of electrical connectors and the second common mode choke 106 may be associated with the first pair 56 of electrical connectors. The first common mode choke 104 and the second common mode choke 106 may reduce noise, or otherwise improve a signal to noise ratio.

In some cases, using separate common mode chokes 104, 106 for each of the pairs 56, 58 of electrical connectors can avoid issue with possible crosstalk between channels, with the pair 56 of electrical connectors representing a channel and the pair 58 of electrical connectors representing another channel. In some cases, a single common mode choke 104 may be used for both of the pairs 56, 58 of electrical connectors. In some cases, coaxial cables may be used instead of twisted wire pairs in order to reduce possible crosstalk. In some cases, concerns regarding possible crosstalk may be reduced or even eliminated by ensuring that the two imaging modalities (with one imaging modality corresponding to the first imaging device 50 and another imaging modality corresponding to the second imaging device 52) are time division multiplexed, meaning that only one imaging modality is active at a time.

In some cases, a compression spring 108 may apply force to the ferrule 100. In some cases, a thrust bearing 110 may reduce friction and wear. Inclusion of the compression spring 108 and the thrust bearing 110 means that the connector assembly 48 may be considered as being spring-loaded in order to maintain physical contact between the optical ferrules. The compression spring 108 is able to apply force to the PCB 84, meaning that the electrical pins 86 that are secured to the PCB 84 are also able to translate. This means that both the electrical pins 84 and the ferrule 100 are able to translate. In some cases, this allows for an electrical and optical connection in which the optical fiber cable does not have to bend, either in the medical device 10 or in the motor drive unit 62. This reduces the risk of reducing optical transmission and/or damaging the optical fiber. This also reduces complexity, by not requiring axial movement of components within the motor drive unit 62.

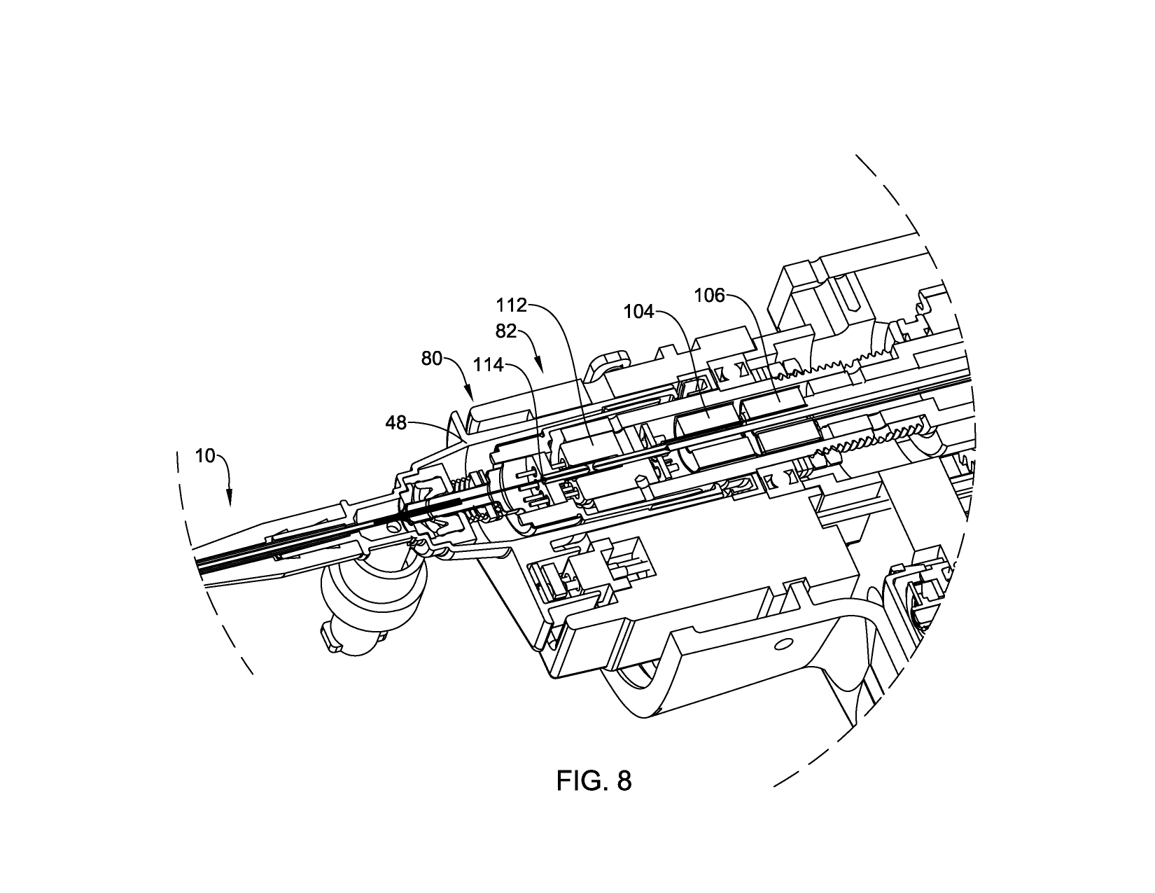

In some cases, a connector block 112 (FIG. 8) may be positioned in front of the PCB 90 in order to help align each of the pin receptacles 92 to receive the electrical pins 86. While not noticeably visible, in some cases, the connector block 112 includes a separate aperture for each of the pin receptacles 92 as well as a centrally located aperture to align a fiber optic ferrule 114 extending through the connector block 112. In some cases, the ferrules 100 and 114 may be polished to a slight convex shape in order to ensure that the fiber cores are in physical contact. This minimizes the gap and maximizes optical transmission between the ferrules 100 and 114.

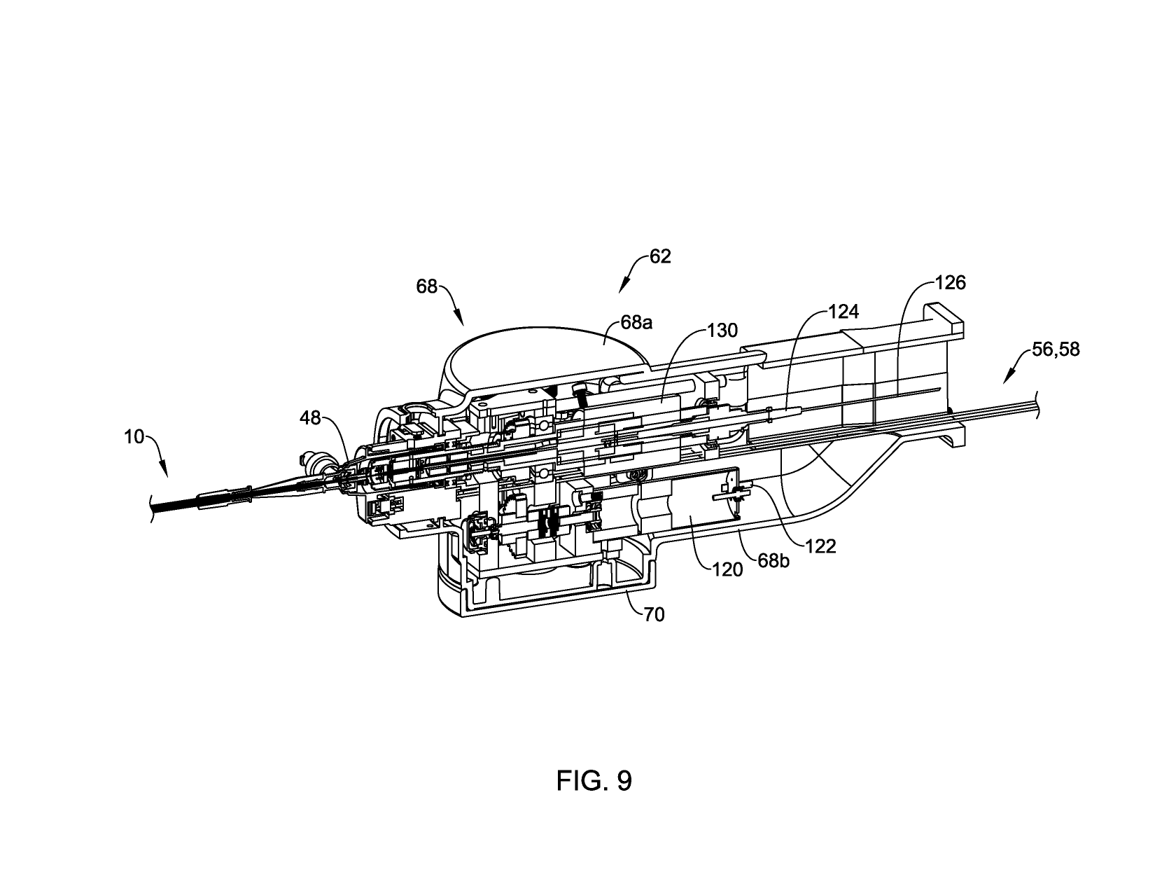

As can be seen in FIG. 9, the motor drive unit 62 includes a rotary motor 120. In some cases, the rotary motor 120 is disposed within the control unit housing 68 such that the rotary motor 120 is located outside of the base section 70 of the control unit housing 68. In some cases, the rotary motor 120 may have an effective length, particularly when including electrical connectors 122, that is greater than an inner diameter of the base section 70 of the control unit housing 68, meaning that the rotary motor 120 may not fit within the base section 70 of the control unit housing 68. In some cases, positioning the rotary motor 120 as shown allows for the base section 70 to be sized to be used with an existing sled 66, for example.

In some cases, the motor drive unit 62 may also include an optical rotary joint (e.g., a fiber optic rotary joint) 124. The optical rotary joint 124 may be coupled with a fiber optic cable 126. In some cases, the motor drive unit 62 may include a component 130. In some cases, the component 130 may include four liquid metal slip rings 132 that are adapted to make sliding contact with a rotating elements within the component 130 (not shown) in order to capture electrical signals from the rotating elements.

The materials that can be used for the various components of the medical device 10 (and/or other devices disclosed herein) and the various tubular members disclosed herein may include those commonly associated with medical devices. For example, the medical device 10 and/or other components thereof be made from a metal, metal alloy, polymer (some examples of which are disclosed below), a metal-polymer composite, ceramics, combinations thereof, and the like, or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), high-density polyethylene, low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

In at least some embodiments, portions or all of the medical device 10 may also be doped with, made of, or otherwise include a radiopaque material. Radiopaque materials are understood to be materials capable of producing a relatively bright image on a fluoroscopy screen or another imaging technique during a medical procedure. This relatively bright image aids the user of the medical device 10in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the medical device 10 to achieve the same result.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. An intravascular imaging system, comprising:
an imaging catheter;
a first imaging device translatable and rotatable within the imaging catheter;
a second imaging device translatable and rotatable within the imaging catheter;
a first connector assembly disposed adjacent to a proximal end region of the imaging catheter, the first connector assembly being configured to mechanically connect the imaging catheter to a control unit;
wherein the first connector assembly comprises:
a first pair of electrical connectors for electrically connecting the first imaging device to the control unit; and
a second pair of electrical connectors for electrically connecting the second imaging device to the control unit.

2. The intravascular imaging system of claim 1, further comprising an optical imaging device that is translatable and rotatable within the imaging catheter.

3. The intravascular imaging system of claim 1, wherein the imaging catheter further comprises an optical imaging device.

4. The intravascular imaging system of claim 3, further comprising an optical fiber cable extending between the optical imaging device and the first connector assembly.

5. The intravascular imaging system of claim 4, wherein the first connector assembly further comprises a ferrule and a connecting sleeve disposed about the ferrule.

6. The intravascular imaging system of claim 1, further comprising a control unit.

7. The intravascular imaging system of claim 6, wherein the control unit comprises a second connector assembly adapted to mechanically and electrically engage the first connector assembly.

8. The intravascular imaging system of claim 7, wherein:
the first connector assembly comprises a plurality of connector pins;
the second connector assembly comprises a plurality of pin receptacles, each of the plurality of pin receptacles adapted to receive a corresponding one of the plurality of connector pins.

9. The intravascular imaging system of claim 6, further comprising a second fiber optic cable extending through the control unit.

10. The intravascular imaging system of claim 6, wherein the control unit comprises a control unit housing.

11. The intravascular imaging system of claim 10, wherein the control unit housing comprises a base section adapted to secure the control unit to a sled.

12. The intravascular imaging system of claim 11, wherein the control unit further comprises a rotary motor that is disposed within the control unit housing such that the rotary motor is outside of the base section of the control unit housing.

13. The intravascular imaging system of claim 1, wherein the control unit comprises:
a first common mode choke associated with the first pair of electrical connectors; and
a second common mode choke associated with the second pair of electrical connectors.

14. An intravascular imaging system, comprising:
an imaging catheter;
an imaging core translatable and rotatable within the imaging catheter;
a connector assembly disposed adjacent to a proximal end region of the imaging catheter, the connector assembly being adapted to mechanically connect the imaging catheter to a control unit;
wherein the connector assembly comprises:
a first pair of electrical connectors for electrically coupling a first imaging device to the control unit;
a second pair of electrical connectors for electrically coupling a second imaging device to the control unit; and
an optical connector for optically coupling an optical imaging device to the control unit.

15. The intravascular imaging system of claim 14, wherein the imaging core comprises:
the first imaging device;
the second imaging device; and
the optical imaging device.

16. The intravascular imaging system of claim 14, wherein the control unit comprises:
a first common mode choke associated with the first pair of electrical connectors; and
a second common mode choke associated with the second pair of electrical connectors.

17. The intravascular imaging system of claim 14, wherein the control unit further comprises:
a control unit housing having a base section; and
a rotary motor that is disposed outside of the base section of the control unit housing.

18. An intravascular imaging system, comprising:
an imaging catheter;
a control unit including:
a control unit housing;
a control unit housing base section adapted to be releasably secured to a sled; and
a rotary motor disposed within the control unit housing outside of the control unit housing base section;
a connector assembly disposed adjacent to a proximal end region of the imaging catheter, the connector assembly being configured to mechanically connect the imaging catheter to the control unit;
wherein the connector assembly comprises:
a first pair of electrical connectors for electrically connecting a first imaging device to the control unit; and
a second pair of electrical connectors for electrically connecting a second imaging device to the control unit.

19. The intravascular imaging system of claim 18, wherein the rotary motor has a length that is greater than a diameter of the control unit housing base section.

20. The intravascular imaging system of claim 18, wherein the control unit comprises:
a first common mode choke associated with the first pair of electrical connectors; and
a second common mode choke associated with the second pair of electrical connectors.
